(19) United States
(12) Patent Application Publication
(10) Pub. No.: US 2026/0311324 A1
(43) Pub. Date: Oct. 8, 2026

(54) A METHOD AND SYSTEM FOR PROCESSING FLUOROSCOPIC IMAGES TO RECONSTRUCT A PATH OR NETWORK

(71) Applicant: MEDICAL ISIGHT (UK) LTD, London (GB)

(72) Inventor: Philip Pratt, London (GB)

(21) Appl. No.: 19/135,880

(22) PCT Filed: Dec. 7, 2023
(86) PCT No.: PCT/GB2023/053161

§ 371 Date: Jun. 5, 2025

(30) Foreign Application Priority Data
Dec. 9, 2022 (GB) 2218532.6

(51) Int. Cl. G06T 7/00; G06T 7/12; G06T 7/246

(52) U.S. Cl. CPC G06T 7/0012; G06T 7/12; G06T 7/246; G06T 2207/10121; G06T 2207/30021

(57) ABSTRACT

A computing system is configured to receive first and second two-dimensional fluoroscopic images acquired at first and second orientations. Each of the images includes a representation of a network in a subject. The network may comprise, for example, an instrument such as a guidewire or an anatomical feature such as a respiratory system, For each image, the representation of the network in that image is divided into a sequence of steps. For each image, a determination is made, for each step in the sequence of steps, of a corresponding back projection to an X-ray source used to acquire the fluoroscopic images. This determination generates a first sequential set of back projections for the first image and a second sequential set of back projections for the second image. A three-dimensional path of the network in the subject is now reconstructed based on a sequence of pairs. Each pair in the path comprises a first back projection from the first sequential set and a second back projection from the second sequential set. The pairs are selected according to the distance of closest approach between the first back projection and the second back projection. A computer-implemented method for performing an analogous reconstruction of a network is also provided.

### FIELD

The present application relates to a method and computer system for processing fluoroscopic images in the context of medical imaging to reconstruct a path or network, for example, a path of a guidewire.

### BACKGROUND

It is common in medical procedures to insert a tool into a luminal network of a patient to perform a medical task or intervention such as removal of a blockage, endoscopy, biopsy, diagnosis, therapeutic action, and so on. In such procedures, the tool is navigated via the luminal network to the desired location at which the medical task is to be performed.

It is important that the tool is tracked within the body so the clinician is able to navigate the tool to a desired location. For example, in the case of a branched network such as blood vessels which have many junctions, the clinician must be able select the correct path for the tool at each junction in order to reach the intended destination. The clinician must also be able to tell when the tool has arrived at the desired location (rather than over-shooting or under-shooting).

In some cases, a luminal network is externally accessible for the insertion of a medical tool, e.g. through oral insertion. In other cases, such as for accessing blood vessels, some form of incision is required. It is generally desired to minimise the size of an incision to support navigation and operation of the medical tool. Limiting an incision in this manner may help to reduce the duration of an operation and allows for a quicker recovery by the patient. However, limiting the size of an incision may prevent a clinician from using direct vision of a tool when navigating the tool through the luminal network.

The use of medical imaging to support a medical procedure is widespread. Such medical imaging may be performed externally to the body (such as most X-ray imaging or some ultrasound) or internally to the body (such as endoscopy or some ultrasound). The use of external medical imaging may support a clinician in navigating a tool through a luminal network while avoiding or reducing the need for any incision into a patient.

A wide variety of medical imaging systems are known, including systems such as magnetic resonance imaging (MRI) and computed tomography (CT) X-ray imaging.

Although such imaging systems are very powerful and are able to produce detailed 3-dimensional (3D) images, they are mainly used in a pre (or inter) operative context rather than in an intra-operative context. There are a number of reasons why 3D MRI and CT imaging are less suited to use in an intra-operative context. For example, the 3D imaging system may be physically obtrusive, and hard to reconcile with the locations of surgical staff around a patient. Such 3D imaging systems are also a valuable resource, and it may not be cost-effective to tie up such a system for the duration of a medical operation (especially if the imaging is only used for a portion of the medical operation). Another consideration, particular with regard to CT imaging, is to reduce the exposure of both surgical staff and the patient to X-ray radiation.

It is known that 3D images produced by inter-operative imaging may not be completely accurate with respect to the current state of a patient during a subsequent surgical operation. For example, a patient might experience some physiological change between the acquisition of a 3D image and the time of the surgery. The patient might also have a different pose for a medical operation compared with the pose for the 3D imaging (such as being on one side rather than lying on his/her back). This can lead to some changes in the orientation and shape of internal soft organs. Furthermore, the presence of a medical tool inserted intra-operatively into a patient can also distort the internal soft organs.

Accordingly, other forms of imaging have been developed to provide real-time imaging in an intra-operative context. These other forms of imaging are typically quicker and less obtrusive than pre-operative MRI or CT imaging. A common approach for performing such intra-operative imaging is referred to as fluoroscopic imaging, which is another form of X-ray imaging. Fluoroscopic imaging involves obtaining one or more 2-dimensional (2D) images, each such image representing an X-ray projection through an object being imaged. In many cases, a pair of fluoroscopic images may be acquired taken from two different orientations (as discussed in more detail below). In some implementations (procedures), a time sequence of such fluoroscopic images may be obtained, while other implementations may utilise just a single set of one or more fluoroscopic images taken at a particular time.

X-ray images are generally formed by transmitted radiation (in contrast to optical images, which are generally formed by reflected light). In particular, an X-ray imaging system typically has an X-ray source to provide a collimated beam of X-rays directed at an object of interest. An X-ray image detector is located behind the object, facing back towards the X-ray source.

The X-ray source can generally be regarded as forming an X-ray shadow of the object on the image detector. However, whereas optical shadows tend to have a sharp (binary) contrast between black, when a solid is located between the X-ray source and the image detector, or white, when no such intervening object is present, X-rays have a much greater power to penetrate through material such as soft tissue in the human body. The intensity of X-rays received by the image detector therefore corresponds to the original (source) X-ray intensity reduced by the cumulative absorption of X-rays along the path from source to detector.

One way of navigating a tool to a desired location is first to navigate a guidewire to this location. The guidewire is generally relatively small in cross-section and easy to direct along a particular path, thereby assisting in navigation of the guidewire to the desired location. Once the tip (distal end) of the guidewire has reached the desired location, one or more tools for performing a given medical task (imaging, biopsy, drug release, ablation, etc) can be mechanically coupled to the guidewire to allow such tools to be inserted along the same path as the guidewire to the desired location.

Fluoroscopic images may be acquired during navigation of the guidewire to help ensure that the guidewire progresses towards and then arrives at the desired location. As noted above, each fluoroscopic image is a projection of an object onto a flat surface. In practice, the projected position (shadow) of the guidewire can be identified fairly easily in fluoroscopic images since the guidewire is usually formed of material (e.g. metal) having high X-ray absorption. A single fluoroscopic image does not allow the full three-dimensional path of the guidewire to be determined (reconstructed). However, the 3D path of the guidewire becomes more accessible if multiple fluoroscopic images are obtained using known, different orientations of the fluoroscopic imaging device. Most commonly only two fluoroscopic images are obtained (to minimise X-ray exposure to the patient) and these two images are processed to perform reconstruction of the 3D path of the guidewire.

There are some known techniques available for this reconstruction of the 3D path of a guidewire from fluoroscopic imaging, see for example:

“THREE-DIMENSIONAL GUIDE-WIRE RECONSTRUCTION FROM BIPLANE IMAGE SEQUENCES FOR INTEGRATED DISPLAY IN 3D VASCULATURE”, S. A. M. BAERT; E. B. VAN DE KRAATS; T. VAN WALSUM; M. A. VIERGEVER; W. J. NIESSEN, IEEE TRANSACTIONS ON MEDICAL IMAGING (VOLUME: 22, ISSUE: 10, October 2003) 29 Sep. 2003;

“3D GUIDE WIRE RECONSTRUCTION FROM BIPLANE IMAGE SEQUENCES FOR 3D NAVIGATION IN ENDOVASCULAR INTERVENTIONS”, S. A. M. BAERT, E. B. VAN DER KRAATS, AND W. J. NIESSEN, IMAGE SCIENCES INSTITUTE, UNIVERSITY MEDICAL CENTER UTRECHT, SPRINGER-VERLAG BERLIN HEIDELBERG 2002;

“THREEDIMENSIONAL CURVILINEAR DEVICE RECONSTRUCTION FROM TWO FLUOROSCOPIC VIEWS”, CHARLOTTE DELMAS, MARIE-ODILE BERGER, ERWAN KERRIEN, CYRIL RIDDELL, YVES TROUSSET, ET AL., SPIE, MEDICAL IMAGING 2015 IMAGE-GUIDED PROCEDURES, ROBOTIC INTERVENTIONS, AND MODELING, FEB 2015, SAN DIEGO, CA;

“MEDICAL TOOL TRACKING IN FLUOROSCOPIC INTERVENTIONS, NEW INSIGHTS IN DETECTION AND TRACKING OF TUBULAR TOOLS”, TIM HAUKE HEIBEL 23 Sep. 2010; and

“DETECTION AND 3D LOCALIZATION OF SURGICAL INSTRUMENTS FOR IMAGE-GUIDED SURGERY”, IRINA BATAEVA, MAY 2021.

See also US 2009/279767 “System for 3-Dimensional Medical Instrument Navigation” and US 2020008885 “Determining a Suitable Angulation and Device”.

Some of the above documents adopt an epipolar approach for performing a guidewire reconstruction from two fluoroscopic images. However, such an approach typically involves making an arbitrary selection of one fluoroscopic image rather than the other fluoroscopic image as a starting point, and hence may not provide the most consistent and reproducible outcome.

### SUMMARY

The invention is defined by the appended claims.

In some implementations, a computing system is configured to receive first and second two-dimensional fluoroscopic images acquired at first and second orientations. Each of the images includes a representation of a guidewire in a subject. For each image, the representation of the guidewire in that image is divided into a sequence of steps. For each image, a determination is made, for each step in the sequence of steps, of a corresponding back projection to an X-ray source used to acquire the fluoroscopic images. This determination generates a first sequential set of back projections for the first image and a second sequential set of back projections for the second image. A three-dimensional path of the guidewire in the subject is now reconstructed based on a sequence of pairs. Each pair in the path comprises a first back projection from the first sequential set and a second back projection from the second sequential set. The pairs are selected for having a low distance of closest approach between the first back projection and the second back projection. A computer-implemented method for performing an analogous reconstruction of a guidewire path is also provided.

In some implementations, a computing system is configured to receive first and second two-dimensional fluoroscopic images acquired at first and second orientations. Each of the images includes a representation of a network in a subject. For each image, the representation of the network in that image is divided into a sequence of steps. For each image, a determination is made, for each step in the sequence of steps, of a corresponding back projection to an X-ray source used to acquire the fluoroscopic images. This determination generates a first sequential set of back projections for the first image and a second sequential set of back projections for the second image. A three-dimensional path of the network in the subject is now reconstructed based on a sequence of pairs. Each pair in the path comprises a first back projection from the first sequential set and a second back projection from the second sequential set. The pairs are selected for having a low distance of closest approach between the first back projection and the second back projection. A disambiguation may be performed by comparing the network to a pre-operative image. A computer-implemented method for performing an analogous reconstruction of a network path is also provided.

The network may comprise a linear path (track) or a branched set of paths such as a tree network, or any other suitable form of network. The network may correspond to an anatomical feature, such as the cardiovascular system, the respiratory system, or the digestive system, or to a medical instrument, such as a guidewire, an endoscopic or laparoscopic tool, or a catheter.

### BRIEF DESCRIPTION OF THE DRAWINGS

Various examples and implementations of the invention will now be described in more detail by way of example only with reference to the following drawings:

FIG. 1 is a schematic diagram showing the use of a C-arm fluoroscope to perform X-ray imaging of a subject.

FIG. 2 is a schematic diagram showing a back projection from the image detector to the X-ray source for the C-arm fluoroscope of FIG. 1.

FIG. 3 provides first and second fluoroscopic images obtained using a phantom and including a guidewire.

FIG. 4 is a schematic diagram showing an example of back projections from a fluoroscopic image such as shown in FIG. 3.

FIG. 5 is a schematic diagram showing an example of the mutual intersections of back projections from first and second fluoroscopic images such as shown in FIG. 3.

FIG. 6 is a schematic diagram showing the use of a table of intersection distances to perform guidewire reconstruction as described herein.

FIG. 7 is a schematic diagram showing the same table of intersection distances as FIG. 6, but with high distance values filtered out, and a proposed path for guidewire reconstruction indicated.

FIG. 8 is a schematic diagram generally showing the same table of intersection distances as FIG. 7, but indicating nodes and edges between the nodes, together with a proposed path for guidewire reconstruction.

FIG. 9 is a schematic diagram showing an example of the intersection of two sets of back projections according to the approach described herein.

FIG. 10 shows views from two different orientations (a) and (b) of potential paths determined using the approach described herein.

FIG. 11 is a flowchart shown an example of the approach described herein for reconstructing the path of a guidewire.

FIG. 12 a schematic diagram showing an example of back projections from first and second fluoroscopic images (analogous to those shown in FIGS. 3 and 5) for use in tracking a network in a patient.

FIG. 13 is also schematic diagram showing an example of back projections from first and second fluoroscopic images for use in tracking a network in a patient. The network of FIG. 13 is the same as the network of FIG. 12, but the fluoroscopic images have been acquired from different locations and orientations.

FIG. 14 is a flowchart shown an example of the approach described herein for reconstructing a network in a subject.

### DETAILED DESCRIPTION

By way of overview, a system and method are described herein which may be used, inter alia, to perform a 3D reconstruction of the length (path) of a guidewire through a patient body using two fluoroscopic images obtained at different orientations (for example, frontal and lateral). This 3D reconstruction involves determining a 3D geometry for the guidewire using two sets of back projections, one set defined for each fluoroscopic image, and the intersections between these two sets of back projections.

In particular, for each fluoroscopic image, three-dimensional locations are known for (i) the X-ray source, and (ii) the X-ray image detector. Therefore, if the guidewire (more accurately, the shadow of the guidewire) is observed at a given location within a fluoroscopic image, then a unique line (back projection) can be defined between the X-ray source and the given location on the X-ray image detector. It is then known that the portion of the guidewire shown in the given location lies somewhere along this unique line.

If we consider first the tip of the guidewire seen in both the first and second fluoroscopic images, then we have two three-dimensional lines, one from each image. The tip of the guidewire must lie along both of these lines. Accordingly, the two three-dimensional lines must intersect, at least approximately- and this direct (exact) intersection represents the three-dimensional location of the tip of the guidewire.

However, the guidewire is relatively featureless (except at its tip). This means that if we select a point along the guidewire shown in a first image, it is generally not possible to immediately identify where the same point of the guidewire is shown in the second image.

Accordingly, unlike for the tip, we cannot identify two lines (one for each image) that are known to arise from the same position along the guidewire, and so cannot directly use such lines to reconstruct the 3D path of the guidewire.

To overcome this limitation, the present approach identifies, for each fluoroscopic image, a closely spaced sequence of points along the projected (imaged) path of the guidewire. For each point in the sequence, a line (back projection) is determined from that point back to the X-ray source. Therefore, for each fluoroscopic image, we end up with an ordered set of lines and it is known that the guidewire is located on a point in the first line of the ordered set, also on a point in the second line of the ordered set and so on.

The 3D shape of the guidewire may now be determined by looking for (exact or near) intersections between the back projections (lines) associated with one image and the back projections associated with the other image. Such intersections generally correspond to points along the length of the guidewire. An optimisation technique may be used to determine the lowest cost (distance) track of the guidewire through 3D space that passes through close intersections of lines from the two fluoroscopy images. The closer an intersection is, the lower the assigned cost/distance for including this intersection in the determination of the 3D reconfiguration of the guidewire. One example of an optimisation algorithm that may be used for determining the track of the guidewire as described herein is Dijkstra's shortest path algorithm (see for example https://en.wikipedia.org/wiki/Dijkstra%27s_algorithm).

One further issue is that the track of the actual guidewire in 3D may bend back on itself, so that a given line from a first fluoroscopic image may closely intersect two (or more) lines from the second fluoroscopic image, in which case each intersection corresponds to a different potential 3D location for the guidewire. The approach of using the optimisation algorithm to determine the track of the guidewire sequentially from the tip is able to provide a disambiguation that associates each intersection with a corresponding position along the guidewire. Once the track of the guidewire has been determined, this may be used (for example) to confirm that the guidewire is correctly positioned, for example with respect to one or more anatomical features identified in pre-operative imaging and/or to determine any distortion in the blood vessels arising from the presence of the guidewire (and potentially to update any pre-operative images such as CT or MRI to include this distortion).

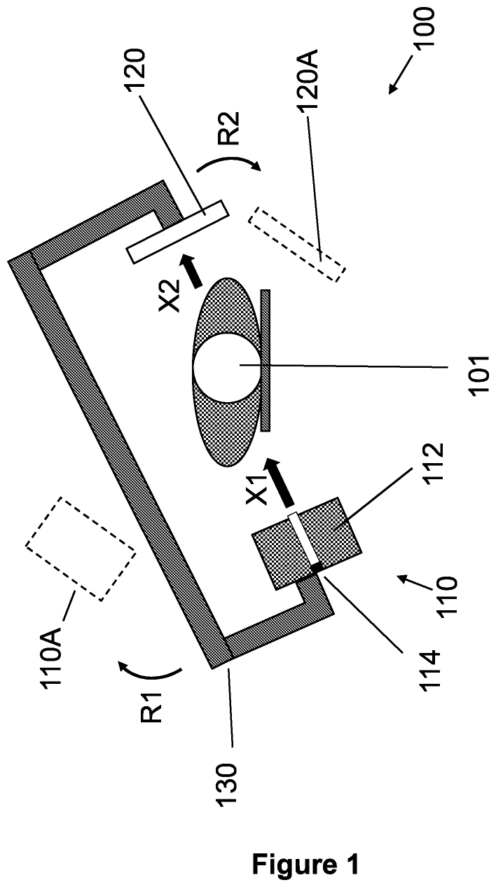

Although the implementation of FIG. 1 described below involves tracking the path of a guidewire, the approach described herein is applicable more generally to identifying a network in a subject. The network may comprise a linear path (track) or a branched set of paths such as a tree network, or any other suitable form of network. The network may correspond to an anatomical feature, such as the cardiovascular system, the respiratory system, or the digestive system, or to a medical instrument, such as a guidewire, an endoscopic or laparoscopic tool, or a catheter. Further information about tracking a network in this manner is described below. Turning now to FIG. 1, this is a schematic diagram showing the use of a C-arm fluoroscope 100 to perform X-ray imaging of a subject or patient 101. The fluoroscope 100 includes an X-ray emitter 110 and an X-ray image detector 120. The X-ray emitter 110 comprises an X-ray source 114 and an X-ray collimator 112 to produce an X-ray beam that is approximately parallel (but with a slight divergence moving away from the X-ray source 114). The X-ray beam from the X-ray emitter 110 is indicated by arrow X1 and is directed at the patient 101. The X-ray beam passes through the patient 101 to produce a transmitted X-ray beam indicated by arrow X2 which is incident upon the X-ray image detector 120. The X-ray emitter 110 and the X-ray image detector 120 are supported on opposing ends of a C-shaped frame 130 (hence the reference to C-arm fluoroscopy).

FIG. 1 shows the C-arm fluoroscope 100 configured to a first orientation or angle. To allow a 3D reconstruction to be performed, a first fluoroscopic image is obtained at the first orientation, and then the frame (and the emitter 110 and detector 120) are rotated to a second orientation to obtain a second fluoroscopic image. In order to move between the first and second orientations, the C-arm 130 may be rotated (for example) about an axis which is perpendicular to page of FIG. 1. In particular example, the rotation is indicated by arrows R1 and R2 such that the X-ray emitter 110 is rotated to the position 110A as indicated by dashed lines, and the X-ray image detector 120 is rotated to the position 120A as again indicated by dashed lines. (Note that modern fluoroscopes typically have two degrees of rotational freedom per C-arm, one as shown in FIG. 1, and another which in effect moves the X-ray emitter 120 into the page and the X-ray detector 120 out of the page (and vice versa). For the present purpose, we focus on the rotational freedom indicated by arrows R1 and R2 in FIG. 1).

The frame 130 holds the X-ray emitter 110 and the X-ray image detector 120 in a fixed, known, relationship to one another. Furthermore, the orientation (rotation angle) of the frame 130 is measured and recorded for each image. Accordingly, for each image, it is known whether the X-ray emitter and detector are located at the positions 110, 120 shown in FIG. 1, or the positions 110A, 120A, or any other rotational position.

It will be appreciated that C-arm fluoroscopes 100 and their use to acquire medical images are, in themselves, well-known to the skilled person. Accordingly, the skilled person will be aware of many additions or modifications to the configuration shown in FIG. 1, which is not intended to be exhaustive, but merely indicative of one possible C-arm fluoroscope 100 (and use thereof) to facilitate understanding of how X-ray images acquired by such a fluoroscope are processed as described herein. As one example of another configuration, we note that in some implementations, there may be two C-arms (having a known relative orientation), whereby two fluoroscopic images may be taken directly with the two C-arms, rather than having a single C-arm which undergoes a rotation between the two images.

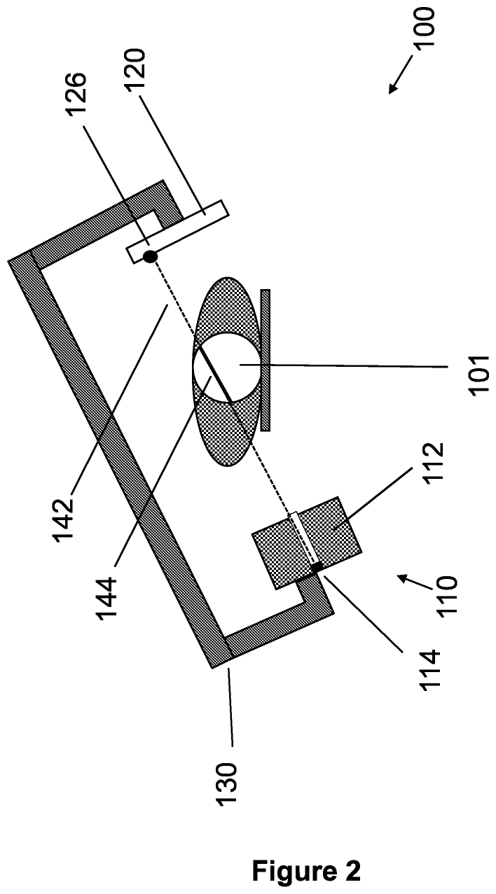

FIG. 2 is a schematic diagram showing an example of back projection from the image detector 120 to the X-ray emitter 110 for the C-arm fluoroscope 100 of FIG. 1. In particular, FIG. 2 shows an image location 126 on the image detector. Also shown is a line (path) 142 which projects back from the image location 126 to the X-ray source 114. A portion of line 142, indicated by reference numeral 144, passes through the head of the patient 101. It will be appreciated that each location 126 on the image detector 120 corresponds to a different path 144 through the head.

The X-ray signal (intensity) recorded at image location 126 is determined by the amount of X-ray absorption along portion 144 of arrow 142. If this absorption is high, the received X-ray intensity at image location 126 will be low (in strong shadow), whereas if the absorption is low, the received X-ray intensity at image location 126 will be relatively high (in light or no shadow).

In the case where a guidewire has been inserted into the patient (not shown in FIG. 2), and the path of the guidewire intersects or crosses portion 144, then the received X-ray intensity will be low, since a guidewire is typically made of a material (e.g. metal) which is strongly absorbing in X-rays. Note however that medical X-ray images are generally presented in negative format, so that a low intensity of received X-rays appears as bright white in the resulting X-ray image, and vice versa for a higher intensity of received X-rays, which appears relatively dark in the resulting X-ray image.

The accuracy of the back projection from an image location 126 to the X-ray emitter is limited by the X-ray beamwidth produced by the collimator 112. In particular, there is a point spread function associated with the X-ray image location 126. The narrower the collimator 112, the tighter the point spread function on the image detector 126, which then allows the back projection path 142 (and hence portion 144) to be determined with greater accuracy.

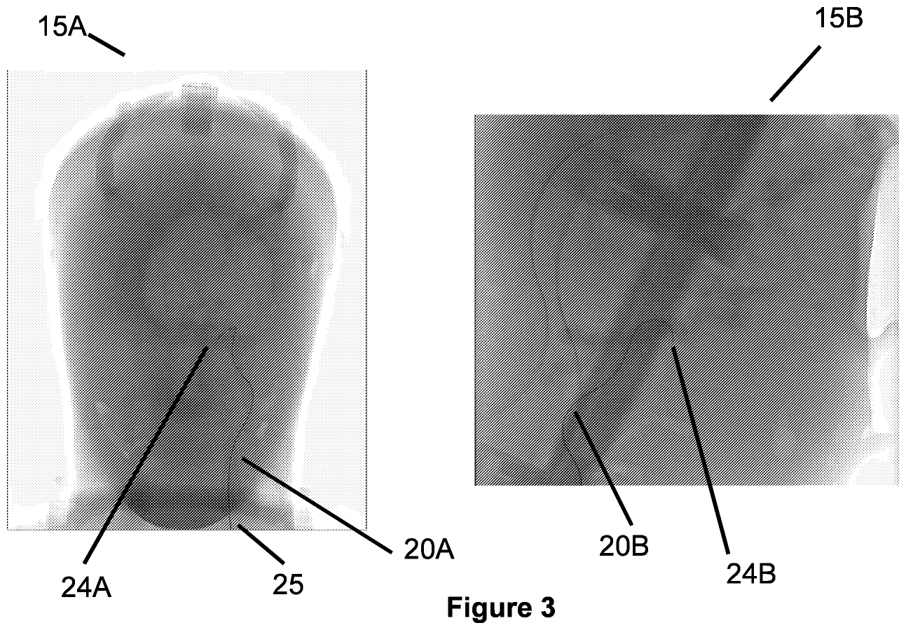

FIG. 3 provides first and second fluoroscopic images obtained from a phantom which is a physical model that may be used, inter alia, for developing and testing technology for use in medical procedures. The phantom shown in FIG. 3 corresponds to a head, but it will be appreciated that the approach described herein for guidewire reconstruction is not limited to use with the head, but rather may be applied to a guidewire reconstruction in any part of the body.

The left-hand image in FIG. 3 will be referred to herein as the first image 15A while the right-hand image in FIG. 3 will be referred to herein as the second image 15B. It will be understood that the terminology first and second images is not intended to indicate the acquisition order for the images-the left image 15A may have been obtained before or after the right image 15B. The first and second images 15A, 15B are taken as a pair, one after the other, with the fluoroscope 100 in respective first and second positions (as discussed above in relation to FIG. 1). In particular, the images 15A, 15B may be taken in quick succession with the head (or phantom) maintained in a constant position across the two images, but with the fluoroscope 100 moved between the two exposures to provide different views (projections) of the head.

The first (left) image 15A has been acquired from a face-on (frontal) perspective while the second (right) image 15B is a lateral view. A guidewire projection image (representation) 20A can be seen as entering the head from the neck at location 25 (which is the edge of the field of view for image 15A) and extending about half-way up through the head in a curved path to the guidewire tip image 24A (distal end) of the guidewire. The guidewire projection image 20A passes along simulated blood vessels in the phantom. The second (right) image 15B also shows a guidewire projection image 20B, including guidewire tip image 24B. it will be appreciated that guidewire projection image 20A and guidewire projection image 20B are two images of the same guidewire taken at different orientations of the fluoroscope (likewise for guidewire tip images 24A, 24B).

Some of the structure visible in FIG. 3 relates to the construction of the phantom, rather than any (simulated) anatomy, and hence can be considered as artificial. For example, the right image 15B includes a pair of lines upper left, however, these are caused by curvature of the erspex structure of the phantom and would not appear in an image of a biological, e.g. human, subject 101.

As described above, in a practical application, the fluoroscope 100 itself tracks the orientation of the X-ray emitter 110 and X-ray image detector 120. The view directions (such as for images 15A, 15B) may then be determined and provided automatically by the fluoroscope 100. It is also possible to determine the view direction for each image 15A, 15B by comparing the structure in the two images-this approach may be adopted, for example, if the calibration information from the fluoroscope is not available.

Note also that the scaling of images 15A, 15B may vary from one image to the other. For example, in the orientation for (say) image 15A, the image detector may be closer to the head than in the orientation for image 15B. The approach for reconstructing the guidewire described herein is able to accommodate such differences in scaling between images 15A, 15B. Nevertheless, if so desired, the images 15A, 15B may be expanded or contracted relative to one another so that they share (approximately) the same scaling (if so desired).

The guidewire projection images 20A, 20B from FIG. 3 indicate the guidewire passing through simulated blood vessels in the phantom, but these (simulated) blood vessels are difficult to see with the X-ray imaging of FIG. 3. The same applies to fluoroscopic imaging of a (real) human subject 101, where the actual blood vessels likewise have low visibility. This provides one motivation for performing reconstruction of the path of the guidewire, since the path of the guidewire can be clearly seen in images 15A, 15B and it is known that the guidewire is contained within blood vessels (so that the latter must follow or trace out the former).

In many cases, a fluoroscopic image does not show the proximal end of the guidewire, rather the guidewire is seen to extend out to the edge of the image (and beyond), as indicated by reference numeral 25 with respect to image 15A. By way of example, in a mechanical thrombectomy within the brain, the guidewire may be inserted through an incision in the groin. This is well outside the field of view of the fluoroscope, which is directed to the portion of the guidewire which passes through the head, since this is the region of primary medical/anatomical interest.

Once a pair of fluoroscopic images 15A, 15B have been obtained with two respective orientations as discussed above, typically in an intra-operative context, for each image 15A, 15B the path of the guidewire projection image 20A, 20B is identified, including the guidewire tip 24A, 24B. This identification is relatively straightforward, given the distinct and prominent appearance of the guidewire projection image 20A, 20B, such as illustrated in FIG. 3, and a variety of known computer-implemented image processing techniques are available in the art for performing such an identification. One such technique looks at the periphery of the image to determine the ingress of the guidewire (as indicated by reference number 25 in FIG. 3), and then determines and follows the progression of the guidewire through the image until the guidewire tip image 24A. 24B is reached. Other implementations are based on the use of artificial intelligence (AI), in which a machine learning ML) system is trained to identify the path of a guidewire in a medical image by providing a suitable training data set of images with the guidewire already identified (labelled). Other approaches can be found in the citations identified in the background section of the present application. The extraction might also be performed by hand by a clinician, or a machine-generated extraction of the guidewire might be subject to confirmation by a clinician.

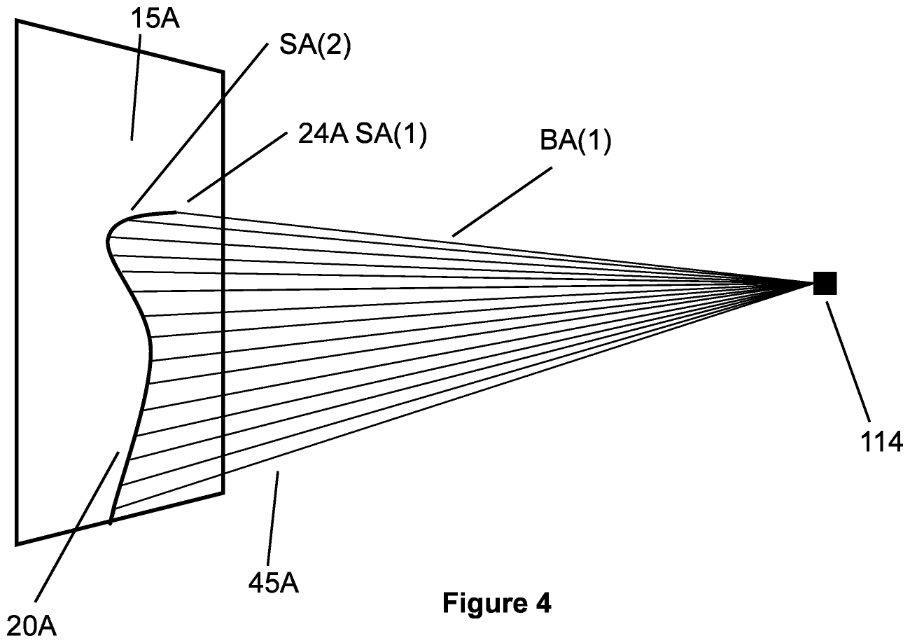

FIG. 4 is a schematic diagram showing an example of back projections from a fluoroscopic image 15A such as shown in FIG. 3. It is assumed in FIG. 4 that the path of the guidewire across the image 15A, including the location of the guidewire tip image 24A, has already been determined as described above. Starting at the identified location of the guidewire tip, a computer-implemented procedure steps along the path of the guidewire as recorded in image 15A. This defines a sequence of step locations along the path which we denote as SA(1), SA(2) . . . SA(i) . . . and so on, where SA(1) corresponds to the initial tip location 24, and the higher the index value (i) the greater the path distance travelled along the guidewire away from the tip location 24.

For each step location SA(i), we determine a corresponding back projection, BA(i), from that location SA(i) back to the X-ray source 114, whereby X-rays emitted from X-ray source 114 that follow the path of back projection BA(i) are incident at the corresponding step location SA(i) in the image 15A recorded by image detector 120. Such a determination is supported because the fluoroscope 100, and in particular the X-ray source 114 and image detector 120 (and image locations obtained by the image detector), have a known and calibrated geometry. This results in a set of back projections 45A being defined with respect to the guidewire path in image 15A,

As described above, the fluoroscopic images 15A, 15B of the guidewire projection images 20A, 20B in effect define the shadows of the real 3D guidewire for each respective orientation of the fluoroscope. For each step location SA(i) in the 2D image of the guidewire projection 20A, there is a corresponding 3D step location RA(i) of the real guidewire. The real guidewire at step location RA(i) gives rise to the step location SA(i) of the guidewire in image 15A. In 3D space, the step location RA(i) is known to lie on the back projection BA(i) path corresponding to SA(i). However, from a single image 15A, it is generally not possible to determine where along this line the real guidewire RA(i) is located, only that it is located somewhere on this line between the X-ray source 114 and the image 15A acquired by image detector 120.

The step size (spacing) between successive locations SA(i) and SA(i+1) is generally chosen to lie in the range of being no smaller than the resolution of image 15A (including any point spread function), but no larger than a size that allows the path of the guidewire projection image 20A as recorded in image 15A to be accurately followed by the stepped locations. Typically the step size is (approximately) uniform along the path of the guidewire projection 20A in image 15A, however, the approach described herein also allows the step size to vary if so desired. For example, a smaller step size might be used in places where the guidewire curvature in image 15A or 15B is relatively high, and a larger step size might be used in places where the guidewire curvature is relatively low (having a lower density of steps for the straighter portions reduces the overall number of back projections, and so can reduce computational complexity).

In practice, the step size may result in hundreds or thousands of step locations along the path of the of the guidewire projection image 20A in image 15A. Accordingly, the maximum index value (i) may be in the range 100 to 100,000, preferably in the range 250 to 10000, but it will be appreciated that the number of steps identified in the guidewire image may depend on the type of medical procedure, the size of the image detector 120, and so on, and hence the above values are provided by way of example only but without limitation.

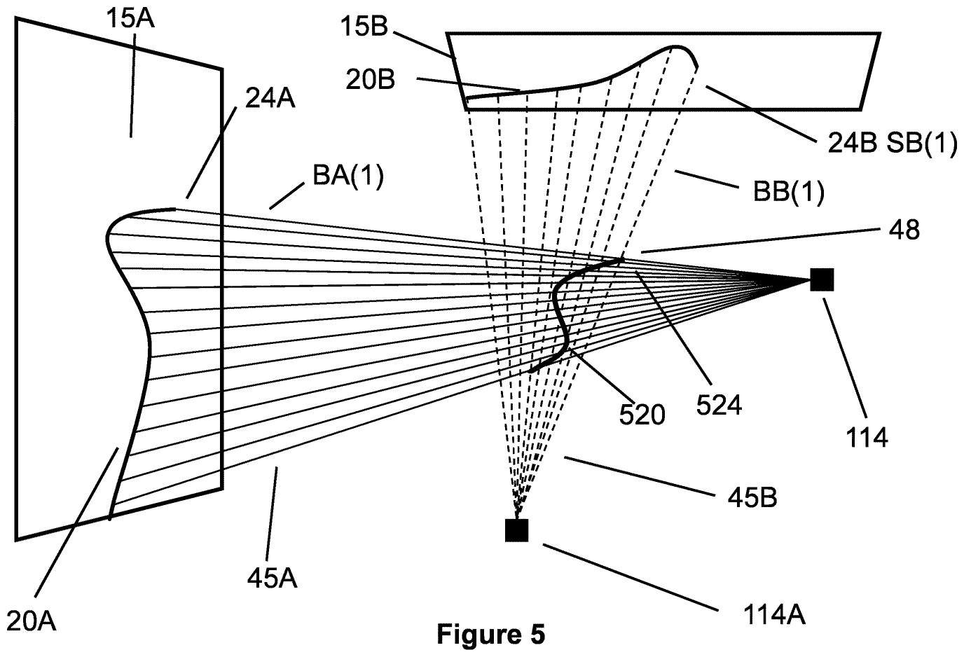

FIG. 5 is a schematic diagram showing an example of the intersection between two sets of back projections 45A, 45B from first and second fluoroscopic images 15A, 15B respectively such as shown in FIG. 3. In particular, the back projections shown in FIG. 4 are also shown in FIG. 5, which further includes back projections 45B from a second image 15B. Image 15B is obtained with the X-ray source at location 114A (as opposed to X-ray source location 114 used to obtain image 15A). Image 15B is processed in substantially the same manner as image 15A as described above to obtain the back projections 45B. In particular, the guidewire projection image 20B including the guidewire tip image 24B are also identified in image 15B, and a set of stepped locations SB(i) are defined along the image of the guidewire starting from the tip. For each step location SB(i), a corresponding back projection BB(i) is determined as shown in FIG. 5.

For each step location SB(i) in the 2D guidewire projection image 20B of image 15B, there is a corresponding 3D step location RB(i) of the real (3D) guidewire, shown schematically in FIG. 5 as guidewire 520. The real guidewire 520 at step location RB(i) is projected onto image 15B and hence gives rise to the corresponding location SB(i) in the path of the guidewire projection image (representation) 20B in image 15B. In 3D space, the step location RB(i) is known to lie on the path of back projection BB(i) corresponding to SB(i). Again, from a single image 15B, it is generally not possible to determine where along this line the real guidewire 520 is located, only that it is located on this line somewhere between the X-ray source 114A and the image 15B acquired by image detector 120.

Note that although FIG. 5 appears to show a larger step size for SB(i) in image 15B compared to the step size for SA(i) in image 15A, this is primarily to simplify FIG. 5 for ease of understanding. In many implementations, the same step size may be used for both images 15A, 15B and associated back projections 45A, 45B, but this is not required for the approach described herein, and a different step size may be used for images 15A and 15B. For example, a difference in step size might be appropriate if images 15A, 15B have a different scaling from one another).

By using the back projections 45A, 45B of both images 15A, 15B, it is possible to determine (reconstruct) the path of the guidewire 520 in three-dimensional space according to the approach described herein. We commence with the guidewire tip 524 of the 3D guidewire 520. The tip 524 is known to lie firstly along back projection BA(1) with respect to image 15A and secondly along back projection BB(1) with respect to image 15B. Accordingly, the 3-dimensional position of the tip 524 must lie at the intersection 48 between BA(1) and BB(1) because this intersection is the only point that lies on both back projections, BA(1) and BB(1).

However, this process cannot be directly extended for use with respect to the next step (increment) along the guidewire projection images 20A, 20B of each respective image 15A, 15B. To consider this further, we define:
a) RΔ is an increment along the 3D guidewire 520
b) AΔ is an increment (step) along the guidewire projection image 20A captured in the image 15A, such as from SA(1) to SA(2)
c) BΔ is an increment (step) along the guidewire projection image 20B captured in the image 15B, such as from SB(1) to SB(2)
d) θA is the angle between (i) the direction of the increment RΔ along the 3D guidewire 520 and (ii) a normal to the plane of image 15A.
d) θB is the angle between (i) the direction of the increment RΔ along the 3D guidewire 520 and (ii) a normal to the plane of image 15B.

For the guidewire reconstruction, we initially identify the 3D location of the tip 524 of the guidewire 520 based on the intersection of back projections BA(1) and BB(1), but we do not know the 3D path of the guidewire 520 away from the tip 524. According to the approach described herein, we choose an increment (step) size, AΔ and BΔ, to be sufficiently small such that we can consider each step or increment to represent a straight line segment. For ease of explanation, we also choose to set AΔ=BΔ (however, the present approach for reconstructing a guidewire does not depend on this setting).

As discussed above, the guidewire projection images 20A, 20B in respective images 15A, 15B are formed by projecting the 3-D guidewire 520 onto the image detector 120 in two different orientations, one corresponding to image 15A and the X-ray source position 114, the other to image 15B and the X-ray source position 114B. This leads to a geometrical relationship between the increment RΔ in 3D space and the increments AΔ and BΔ within images 15A, 15B respectively. In particular for image 15A we have RΔ (A)=AΔ/sin θA, while for image 15B we have RΔ (B)=BΔ/sin θA, where RΔ (A) represents the specific increment in the 3D guidewire 520 corresponding to the increment AΔ in image 15A and RΔ (B) represents the specific increment in the 3D guidewire 520 corresponding to the increment BΔ in image 15B.

Since we have assumed that AΔ=BΔ, then RΔ (B)=RΔ (A) only in the specific case that θA=θB—in effect when the increment of the guidewire 520 is in a direction which bisects the positioning of images 15A and 15B (more particularly the positionings of the image detector 120 to obtain these two images 15A, 15B). In the general and most common situation, θA≠θB, so that RΔ (B)≠RΔ (A).

In other words, the location SA(2) in image 15A corresponds to a distance RΔ (A) from the tip 524 of the guidewire, while the location SB(2) in image 15B corresponds to a distance RΔ (B) from the tip of the guidewire 520, where RΔ (B)≠RΔ (A) in the general case. Therefore, while the tip 524 could be found by the direct intersection of BA(1) and BB(1), the back projections BA(2) and BB(2) do not relate to the same distance (increment) along the guidewire 520. Accordingly, in the general case, the back projections BA(2) and BB(2) will not directly intersect with one another, since these two back projections relate to different points along the guidewire 524. (It will be appreciated that while FIG. 5 might appear to show all back projections 45A intercepting with all back projections 45B, this is an artefact of the two-dimensional nature of FIG. 5. In 3D space, back projections BA(2) and BB(2) may have paths which are above or below one another, and hence are able to pass each other without any direct intersection).

This problem can be regarded as a lack of synchronisation between the step locations SA(i) and SB(i), in that there is initial synchronisation for the tip 524 corresponding to SA(1) and SB(1), but for subsequent steps this synchronisation cannot be maintained. If the guidewire 520 had regular markers along its length that showed up in images 15A, 15B, then it would be possible to maintain synchronisation. In particular, we could identify both SA(2) and SB(2) with the first marker, SA(3) and SB(3) with the second marker, and so on. In such an approach, the increments along the guidewire projection image 20A would become variable, likewise the increments along the guidewire projection image 20B, corresponding to changes in the direction and curvature of the guidewire 520 along its path. The back projections BA(2) and BB(2) would then intercept to give the location of the first marker, the back projections BA(3) and BB(3) would intercept to give the location of the second marker, and so on.

Although the use of such markers (or similar) on the guidewire would assist in reconstruction, in practical terms the guidewire 520 is generally designed to be thin and smooth, without external markings or structure, to support ease of insertion along narrow blood vessels. Accordingly, such a guidewire does not provide the markings or structure that might be used to achieve synchronisation between location SA(i) and SB(i) and hence a different approach must be used to reconstruct the path of such a smooth guidewire 520 that does not rely on synchronisation between SA(i) and SB(i).

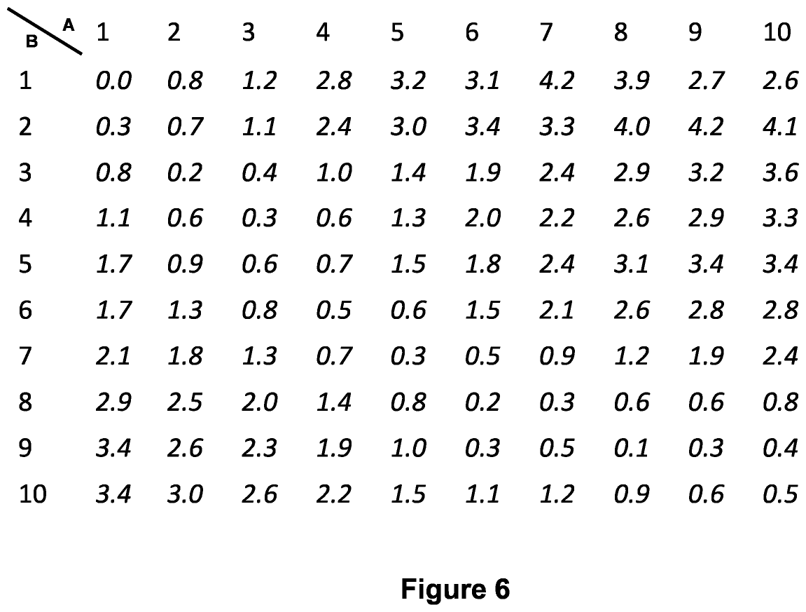

As described herein, the guidewire reconstruction is based on determining, for each back-projection BA(i), the closest approach with each back projection BB(i). This is illustrated in schematic form by the table of FIG. 6. Each column in the table corresponds to a back projection BA(i) from the set 45A, for example, column 1 corresponds to BA(1), column 2 corresponds to BA(2), and so on. Similarly, each row in the table corresponds to a back projection BB(i) from the set 45B, for example, row 1 corresponds to BB(1), row 2 corresponds to BB(2), and so on.

We can specify a particular entry in the table as [j, k] where j represents the column number and k represents the row number. For example, [2, 4] corresponds to column 2, namely BA(2), and row 4, namely BB(4). Each entry in the table relates to the intersection between the two back projections corresponding to that entry. In particular, each table entry records the minimum distance of the closest approach between these two back projections. For example, [2.4] has a value of 0.6 which represents the closest approach between the two back projections BA(2) and BB(4), while [6, 7] has a value of 0.5 and represents the closest approach between the two back projections BA(6) and BB(7). (The distance values given in FIG. 6 are for illustration only with arbitrary units for the distance).

As explained above, each back projection BA(i) corresponds to a location RA(i) along the real 3D guidewire 520 and each back projection BB(i) likewise corresponds to a location RB(i) along the real 3D guidewire 520. However, in the general case RA(i)≠RB(i) because the projection of a given increment RΔ along the 3D guidewire onto images 15A and 15B varies in size according to the orientation of the 3D guidewire 520 relative to the plane of each image 15A, 15B (and the two orientations are chosen to be different to get different views of the guidewire 520 in images 15A, 15B). The only exception to this is for the tip 524 of the guidewire, since this specifically identified point on the guidewire 520 can be directly mapped to both SA(1) and SB(1) in images 15A, 15B respectively. Therefore the intersection value for location [1, 1] in the table has a value of 0.0, indicating a direct intersection—in effect, a closest distance of zero. Note that in practice, the intersection distance is not expected to be exactly zero, due to measurement errors, rounding errors, and so on in the processing to determine the intersection distances.

Nevertheless, if we consider a given position R(x) along the 3D guidewire, this must correspond to some location in the guidewire projection image 20A in image 15A and likewise to some location in the guidewire projection image 20B in image 15B. We express this as R(x) corresponding to locations SA(x1) and SB(x2). If we can determine the locations of SA(x1) and SB(x2) in images 15A and 16B, then the associated back projections, BA(x1) and BB(x2) will intercept at the 3D location R(x). If we can determine the intersection locations for many different values of R(x), this then allows the 3D path of the guidewire 520 to be reconstructed.

In the context of FIG. 6, we select a position SA(i) in image 15A that corresponds to a position (unknown) of R(x) along the 3D guidewire 520, the position R(x) being responsible for the X-ray signal/shadow at position SA(i) in image 15A. We then try to find a position (row) SB(j) in image 15B that likewise corresponds to position R(x) along the 3D guidewire 520, whereby the position R(x) is also responsible for the X-ray signal/shadow at this position SB(j) in image 15B. At this stage, SA(i) is known (selected), but SB(j) and R(x) are not known. However, we do know that since SA(i) and SB(j) both relate to the same portion of the 3D guidewire, their respective back projections should intercept one another, and this intersection then represents the position R(x) for the 3D guidewire 520. This gives us a method of finding SB(j) (and hence R(x)) by looking through the table of FIG. 6 to try to find a position in the second image 15B having a back projection BB(j) that intercepts (or nearly intercepts) the back projection BA(i).

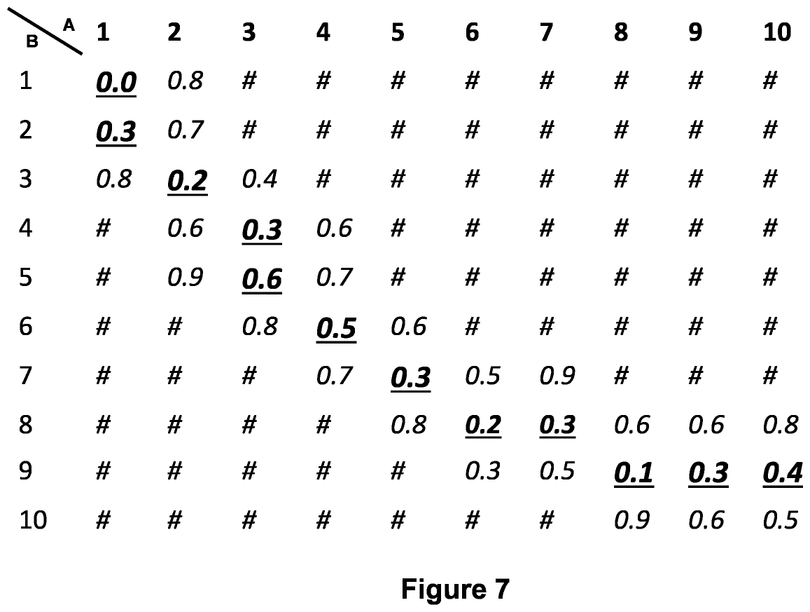

This approach is further illustrated in the table of FIG. 7, which is the same as the table of FIG. 6 but the intersections with a closest distance of over 1 have been marked ‘#’ on the basis that a distance of 1 or more between two back projections is not considered as an exact or near intersection. This threshold may be adjusted according to the circumstances and properties of any given data set. FIG. 7 further shows a path of entries through the table underlined and with green backing. These entries along the path represent pairs of back-projections, one from image 15A (according to the column), and one from image 15B (according to the row), which have a close intersection with one another, indicating that they both correspond to the same distance R(x) along the real 3D guidewire 520 and so can be used for determining the 3D location of R(x).

We can process the entries of FIG. 7 by commencing at the top left corner [1, 1], which we know corresponds to the tip 524 of the real guidewire 520 as explained above.

Accordingly, entry [1, 1] is underlined and shown in bold italics. We now select each column in turn, and step through each row in turn within that column. So remaining within column 1 (corresponding to back projection BA(1)), we next go to entry [1, 2] which has another low intersection value, i.e. low closest distance. We can therefore regard the intersection [1, 2] as corresponding to a progression along the 3D guidewire 520 based on the intersection of back projection BA(1) and BB(2). The intersection [1, 2] is therefore underlined and shown in bold italics. The remaining rows in column 1 have higher intersection values, i.e. a higher closest distance and so do not correspond to a point on the guidewire reconstruction.

We next move to column 2, and step down the row to reach [2, 3] which represents a close intersection. This intersection between back projection BA(2) and back projection BB(3) is therefore considered to provide a further point which lies on the reconstructed path of the guidewire 520. The remaining rows in column 2 are have higher values and so are not used to identify the next point on the guidewire.

The above procedure is repeated for all columns of the table of FIG. 7 to identify the exact or near intersections as described above, and hence to determine a path through the table as shown in FIG. 7. Each identified exact or near intersection represents a position where a back projection 45A from image 15A and a back projection 45B from image 15B both correspond to the same position on the guidewire 520, which is therefore located (reconstructed) at that intersection. Conversely, we could repeat the above procedure for each row (instead of for each column), because the algorithm is symmetric in this respect i.e. the rows and columns could be transposed without changing the outcome.

It can be seen that the general trend of the highlighted path in FIG. 7 is downwards and to the right. This is expected since progressing along the real 3D guidewire 520 also generally progresses along the guidewire projection images 20A, 20B in images 15A, 15B, thereby increasing both the column and row numbers. The path eventually exits the table along the bottom or along the right hand edge according to whether the guidewire 520 first goes out of the field of view of image 15A or image 15B.

The path identification described above in relation to FIGS. 6 and 7 is intended to provide a small-scale version of the guidewire reconstruction based mainly on inspection. However, a more powerful and robust implementation may be used to determine the path through the table of FIGS. 6 and 7, especially given that in a practical situation, the number of back projections 45A, 45B associated with each image might be hundreds or thousands (rather than the 10 rows and columns shown in FIGS. 6 and 7). The more powerful implementation is also able to handle more complex shaping of the guidewire, for example loops, folding over, self-occlusion, and so on (such complex shaping is not included in the above example of FIGS. 6 and 7).

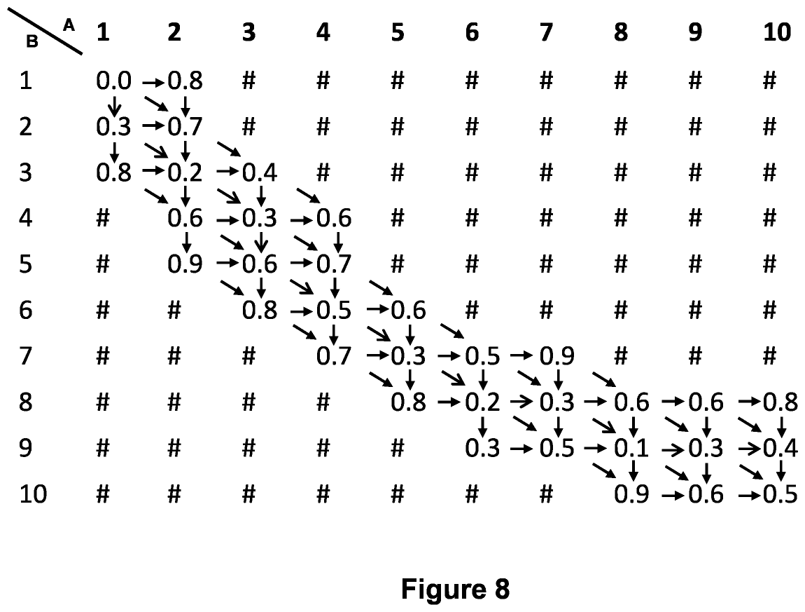

In one approach, the reconstruction of the path of the guidewire is based on a node-edge graphical representation. With reference to FIG. 8, this shows the same data set as depicted in FIGS. 6 and 7. Each intersection of a row and column is considered to represent a node in the graph. Nodes for intersections marked “#” can be discarded again. For each node, one or more edges (shown by the arrows in FIG. 8) are defined which link one node to another node. As discussed above, the path of guidewire 520 must correspond to a sequence of intersections moving down and across the table (graph) of FIG. 8.

Therefore, the edges in FIG. 8 are directed down, right, or a diagonal combination of the two. (Some implementations might not utilise a diagonal edge but rather just rely on down and right edges to track the path).

Each edge in FIG. 8 is directional and extends from an initial (source) node to a destination node. In the example of FIG. 8, the destination node is the next node right, the next node down, or the next node diagonally down and right (according to the structure of the table shown in FIG. 8). Limiting the edges to map between neighbouring nodes in this manner reflects the continuous nature of the guidewire being tracked, hence the path through the nodes is likewise continuous. Note also that the edges flow monotonically from the start at tip 524 in the top left corner to the exit 25 of the guidewire projection image 20A from image 15A (or the exit of the guidewire projection image 20B from image 15B, whichever is first). There are no closed loops in the graph, due to the directionality of the edges, hence the number of possible routes is finite. In other implementations, the graph may be constructed so that the downward, right-moving, and down-right diagonal edges are bidirectional. This bi-directionality gives more options for the selected path, in other words the selection of this path is more general and subject to fewer constraints. Note that the shortest path optimisation ensures that the route does not end up spinning around needlessly.

An optimisation algorithm may be employed to determine the best (lowest cost) route through the graph of FIG. 8, from the top left corner (corresponding to the tip 524) to the exit. As with FIGS. 6 and 7, it will be appreciated that the example of FIG. 8 provided by way of illustration, and is much smaller than a practical application with hundreds if not thousands of back projections. Furthermore, the shape of the guidewire in FIG. 8 is relatively simple to support ease of understanding.

As discussed above, the cost associated with a given route from the tip 524 to the exit may be determined by the summing the intersection distances associated with each node along the route. In particular, the intersection distances represent the distance of closest approach for the pair of back projections corresponding to the node or intersection, one back projection in the pair being taken from set 45A associated with the image 15A, the other back projection in the pair being taken from set 45B associated with the image 15B.

FIG. 8 shows the same path or route as the one highlighted in FIG. 7 by having the edges that define this route shown as−>with a linear arrowhead (whereas the other edges have a solid arrowhead [symbol] ). If we follow the route defined by these edges with a linear arrowhead, the cost (for the portion of the route shown in FIG. 8) is 0.0+0.3+0.2+0.3+0.6+0.5+0.3+0.2+0.5+0.1+0.3+0.4=3.7 Other routes that may be constructed through the table of FIG. 8 (more generally, through the defined graph) have a higher sum of intersection distances along the route. Accordingly, the route specifically indicated in FIG. 8 is considered (selected) to represent the track of the guidewire 520. In some implementations, Djikstra's algorithm may be used to analyse the node-edge configuration to determine the optimal (lowest cost) path through the graph. However, any other suitable optimisation algorithm may be employed. Furthermore, while some implementations may calculate the cost as a simple sum of the intersection distances, the cost may be based on any other suitable function of the intersection distances (such as a sum of squared distances, etc).

Once an optimal route has been determined as above, the track of the guidewire 520 can be reconstructed based on the nodes included in the route. For direct intersections (if any) in the route, the location of the intersection may be taken as being on the track of the guidewire. For near interactions, a suitable location such as midway along the line of shortest distance between the two back projections forming the node may be taken as being (approximately) on the track of the guidewire. Starting at the guidewire tip 524, the path of the guidewire 520 can then be reconstructed based on the locations for the sequence of intersections (nodes) specified in the optimal route.

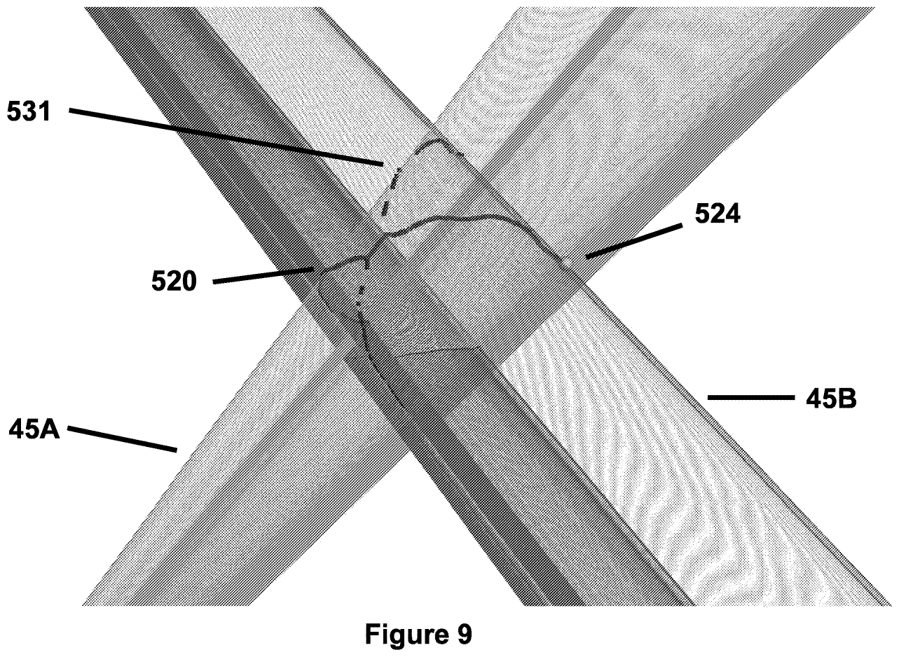

FIG. 9 is a schematic diagram showing an example of the intersection of two sets of back projections 45A and 45B according to the approach described herein. Each of these two sets is formed from approximately 1000 back projections which are nearly parallel with one another but have a slightly divergence as discussed above (since they emanate from the same X-ray source 114). Note that some of the apparent structure in FIG. 9 for back projections 45A and 45B is a display artefact due to the Moiré effect.

FIG. 9 shows the closest intersection for each pair of back projections, whereby each pair comprises one back projection from set 45A and another back projection from set 45B. Pairs for which the closest intersection exceeds a threshold are discounted from FIG. 9 (these pairs correspond to intersections marked #in FIGS. 7 and 8).

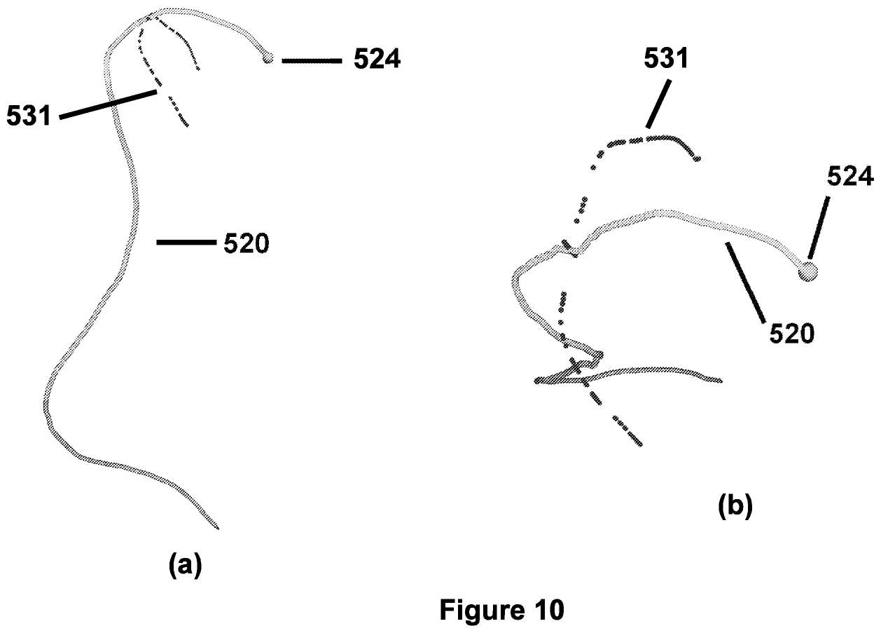

FIG. 9 further shows most of the intersections (pairs); these pairs lie along the reconstructed path of the guidewire 520 which corresponds to the optimal (lowest cost) route of nodes and edges as described above. The path of the guidewire commences at the tip 524. FIG. 9 further shows another apparent route of intersections 531 which also has a relatively low cost. However, the approach described herein is able to perform a disambiguation, in effect discarding potential guidewire path (route) 531 in favour of the proper guidewire path 520. Firstly, potential route 531 does not start (or go near) the tip 524 of the guidewire 520—as discussed above, this location of the tip 524 can be reliably determined, because the tip can be readily identified in both of the fluoroscopic images 15A, 15B. Secondly, the proper guidewire path 520 (terminates on either the bottom or the right hand side of the grid/graph, both of which can be considered as representing going outside the field of view, as discussed above. In contrast, the potential route 531 does not appear to extend this far (see also FIG. 10 discussed below).

It is also noted that the potential route 531 includes relatively large gaps and breaks (as visible in FIG. 9), whereas the proper guidewire path 520 has only small gaps or breaks and so appears to be nearly continuous. The gaps in the proper guidewire path 520 correspond to the intersections associated with the potential route 531 (and vice versa). The gaps in potential route 531 are large, because a significant majority of the intersections are located on the proper guidewire path 520.

FIG. 10 shows the same two routes as FIG. 9, but without the two sets of back projections. In particular, FIG. 10 shows the correct route 520 (light) in combination with the incorrect route 531 (dark). Two different view orientations are shown in images (a) and (b) (note also that the scaling of (a) and (b) is different). FIG. 10 emphasises the relatively large breaks/gaps in incorrect route 531 compared with size of the breaks in the line of the correct route 520 (these breaks are generally too small to be seen in FIG. 10). It can also be seen in FIG. 10 (see especially view (a)) that the incorrect route 531 does not extend close to the far end of the correct route 520, i.e. furthest away from tip 524. The incorrect route 531 therefore peters out prior to reaching the edge 25 of the image 15A or 15B. Accordingly, the approach described herein is readily able to perform a disambiguation regarding the route, discarding potential route 531 in favour of the correct route 520, for example because incorrect route 531 does not start at tip 524, does not extend to the edge of the field of view and/or because the gaps or breaks in the incorrect route are much larger than the breaks in the correct route.

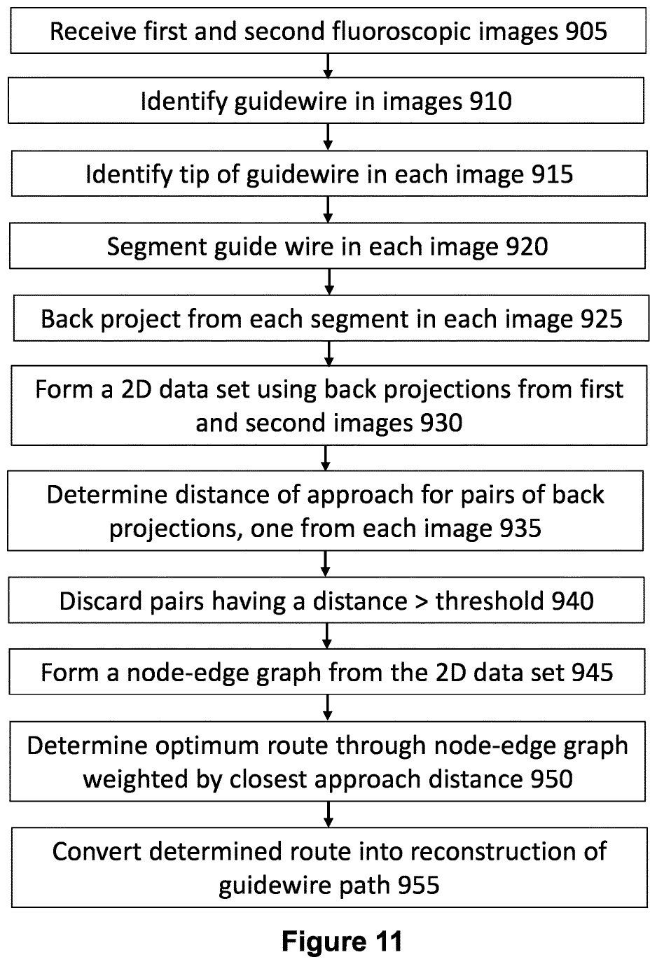

FIG. 11 is a flowchart showing an example of the approach described herein for reconstructing the path of a guidewire 520. The procedure starts (905) with the receipt of first and second fluoroscopic images 15A, 15B. These images are taken by a fluoroscope 100 from two different positions (orientations) at substantially the same time (subject to practical constraints, such as the moving the fluoroscope between the two different positions). The guidewire 520 is maintained at a constant position within the subject (patient) 101 across both images. The fluoroscope also provides calibrated information with regard to the relative positions between the X-ray source 114 and the image detector 120, and also with regard to the shift in orientation between the first and second images.

At operation 910, the path of the guidewire 520 is identified in each of the first and second images 15A, 15B. In other words, a line is detected in each image 15A, 15B which corresponds to the projection or shadow of the guidewire 520 onto the image detector 120 during the acquisition of images 15A, 15B. Each of these lines provides a two-dimensional image or representation of the guidewire projection images 20A, 20B in the respective images 15A, 15B. These representations of the path of the guidewire can be determined using various known algorithms. In some cases, these representations may already have been determined (e.g. using software associated with the fluoroscope) and hence are provided with the first and second fluoroscopic images at operation 905; in such circumstances operation 910 may be omitted.

In a similar fashion, at operation 915 the location (representation) 24A, 24B of the tip 524 of the guidewire 520 is identified in each respective image 15A, 15B. In other words, a point 24A, 24B is detected in each image 15A, 15B which corresponds to the projection or shadow of the guidewire tip 524 onto the image detector 120 during the acquisition of the images 15A, 15B. It will be appreciated that operation 915 may be combined with operation 910, in that as the 2D line or guidewire projection image 20A, 20B corresponding to the projected track of guidewire 520 is determined, this line has one end located in the image 15A, 15B, which corresponds to the tip 24A, 24B, and another end which exits the field of view from images 15A, 15B (see for example exit point 25 for image 15A). Again, it is possible that the position of the tip in images 15A, 15B may already have been determined, e.g. using software associated with the fluoroscope to locate the guidewire, including the tip thereof. This information could then be provided with the first and second fluoroscopic images at operation 905, in which case operations 910 and 915 may both be omitted.

At operation 920, for each image 15A, 15B, the representation or guidewire projection image 20A, 20B of the guidewire is segmented. Typically this segmentation is performed by starting at the tip 24A, 24B of the guidewire and then progressing by successive steps or increments along the 2D representation of the guidewire projection image 20A, 20B. In general, a consistent step size or increment is used throughout this progression. Note that this constant step size relates to the 2D projection of the guidewire as provided by (within) images 15A, 15B. The step size is not constant with respect to the corresponding 3D path of the guidewire 520 because the angle between the 3D path of the guidewire and the plane of images 15A and 15B changes as the guidewire curves through the network of blood vessels. In general, the same step size may be used for each image 15A, 15B, but in some cases a different step size may be chosen for images 15A and 15B, for example if the images 15A, 15B were acquired with a different scaling from one another.

The step size of the segments (increments) is small enough to ensure an accurate sampling and model of the path of the guidewire. In some implementations, the step size is small enough so that the segments may be treated as straight line segments. For example, if we define the curvature of a segment as the angle between a tangent to the initial portion of the segment and a tangent to the final portion of the segment, this might be less than 5 degrees, preferably less than 1 degree to be considered as a straight line. In practical terms, the number of segments in the segmentation may be over 200, preferably over 500, and typically around 1000. Note that the number of segments may vary somewhat between image 15A and image 15B because the 2D projected length of the guidewire projection image 20A, 20B will generally change with viewing orientation. In some implementations, the step size may be adjusted between the two images 15A, 15B so that the same number of segments may be formed for each image 15A, 15B.

At operation 925, for each image 15A, 15B, a back projection is calculated back to the X-ray source 114 from each step or segment in the guidewire projection image (representation) 20A, 20B (based on its position within image 15A, 15B). The information for calculating this back projection is available from the available calibration data, such as may be provided with the fluoroscopic images. Accordingly, operation 925 provides a first set of back projections 45A for image 15A and a second set of back projections 45B for image 15B. The number of back projections corresponds to the number of segments formed for each guidewire projection image (representation) 20A, 20B. (As noted above, the number of back projections 45A, 45B may vary a little between image 15A and image 15B because the projected 2D length of the guidewire will vary according to the viewing angle for image 15A in comparison with the viewing angle for image 15B).

At operation 930, a two-dimensional data set is formed from the two sets of back projections 45A, 45B. In particular, one axis of the data set represents the sequence of back projections formed from image 15A (representing columns in FIG. 8) and the other axis of the data represents the sequence of back projections formed from image 15B (representing rows in FIG. 8). The origin of this data set, i.e. the start of the two axes, corresponds to the tip location 24A, 24B in each image, and progression along each axis then corresponds to progression along the guidewire representations 20A, 20B away from the tip locations 24A, 24B.

The two-dimensional data set can be considered as a grid or array of column-row intersections, for example, a particular location in the array may correspond to the ith back projection from image 15A (column i) and the jth back projection from image 15B (row j). At operation 935, the values to populate this 2D data set are determined. In particular, the value at each intersection (column i, row j) corresponds to the closest distance between the ith back projection from image 15A (column i) and the jth back projection from image 15B (row j). If there is an exact intersection for column i, row j, i.e. a closest distance of 0, then this point of intersection between these two back projections is known to lie on the 3D path of the guidewire 520.

In practice, there are various sources of noise, hence intersections corresponding to locations on the path of the guidewire generally do not have a shortest separation distance of zero. (The noise may come from various sources, such as finite beamwidth of X-rays, limited resolution of the X-ray images, quantisation noise due to the segmentation, other structure such as anatomy in the X-ray images, and so on). Therefore, the 3D path of the guidewire may generally be indicated by low (rather than zero) intersection values.

At operation 940, intersections with high closest distance values, for example, distances above a threshold, are blanked out or discounted. These high value closest distances indicate intersections which do not correspond to the 3D path of the guidewire 520. In practice, a large majority of intersections are discounted in this manner, which greatly simplifies the identification of the 3D path of the guidewire based on the remaining intersections with lower values. This simplification therefore supports quicker calculation of the 3D path of the guidewire 520, which is particularly beneficial in a real-time, intra-operative environment.

In broad terms, the 3D path of the guidewire may be determined by an optimisation procedure which uses closest (nearest) distance as a cost. In other words, if the closest distance for an intersection is relatively low, this corresponds to a low cost, and hence the corresponding intersection is more likely to form part of the 3D path of the guidewire 520.

Note that the cost may directly equal the closest distance, or be some function of closest distance, wherein increasing the closest distance also increases the cost.

There are various techniques available for performing such an optimisation. One such approach involves representing the 2D data set in graph form at operation 945. In this representation, the intersections form nodes in the graph, each node being associated with a cost based on the closest distance value for the corresponding intersection as described above. There are no nodes corresponding to the intersections that have been discounted at operation 940, since these are known not to be on the path of the guidewire. The nodes are linked by edges. Because the 3D path of the guidewire 520 is known to be continuous, the edges permit the route to step from one node to a neighbouring node (according to the layout in the 2D data set, such as illustrated in FIG. 8).

Accordingly, the graph defines a finite set of potential routes (node-edge sequences). Each such route defines a sequence of nodes (intersections) and each route has an associated cost based on the cost of the nodes (intersections) included in the route. At operation 950, an analysis or optimisation is performed to determine the optimal, i.e. lowest cost, route out of the set of potential routes. There are a number of different algorithms available for performing this optimisation, such as using Dijkstra's algorithm (by way of example).

Finally at operation 955, the selected (optimal) path of nodes is used to reconstruct the path of the guidewire 520. In particular, each node relates to the intersection of a first back projection from image 15A with a second back projection from image 15B. We now determine the location of the intersection. The line representing the shortest distance between the first and second back projections is determined. The location of the intersection in this case corresponds to the point halfway along this line, i.e. at the midpoint between the first and second back projections. The locations of the intersections as determined in this manner lie on the path of the guidewire, and hence can be used to reconstruct the path of the guidewire.

As discussed above, the reconstructed path of the guidewire can be used for various purposes, such as checking that the guidewire is correctly located within the subject for performing a planned intervention, and/or for comparing with pre-operative 3D imaging to see if there has been any change or distortion in the path of the blood vessels (such as might be caused by the insertion of the guidewire). The reconstructed path of the guidewire might also be used as a boundary condition to modify the (static) geometry of a vessel network.

It will be appreciated that some of the operations shown in FIG. 11 may be omitted in some implementations. For example, the received first and second images may already have the guidewire identified therein (rather than performing such an orientation at operation 910)—e.g. because the fluoroscope 200 used to acquire the first and second images may also have the facility itself to identify the guidewire in the images. Likewise, discarding pairs having a distance greater than a threshold could be omitted. However, this would significantly increase the number of potential routes and hence may also increase the computational resources involved to determine the optimal route (although this is less of a concern with an efficient algorithm such as Djikstra's algorithm which can discount high cost nodes relatively quickly). In addition, certain operations may be combined, performed in parallel, or performed in a different order. For example, discarding pairs having a distance greater than a threshold could be performed when the 2D data set is first created, or such discarding pairs may not be deleted until the cost corresponding to such a distance has been determined.

The approach for reconstructing the path of a guidewire as described herein may be regarded as global, in that it exploits all the available data deriving from the full length of the 2D guidewire projection images 20A, 20B. In addition, the data from both images 15A, 15B is handled in the same manner, so there is no arbitrary selection of one image ahead of the other image. By taking a global perspective, a disambiguation can be performed, so that path sections which may have a relatively low cost, but which do not reflect the actual path of the guidewire 520, can be readily distinguished and discounted—for example, because they do not start at the tip 524 of the guidewire, because they do not extend the full distance to the exit 25 of the guidewire from the field of view. Accordingly, the approach described herein for reconstructing the path of the guidewire 520 offers various technical benefits compared with existing techniques for performing such a reconstruction.

The approach described above for reconstructing the path of a guidewire can be generalised such as for reconstructing the path of other types of surgical instrument within a subject, or for determining the path (network) of anatomical features within the subject. In this generalised approach, rather than just back-project pixels at certain points along a guidewire, source images (2×2D) comprising actual fluoroscopic images of vessel networks are acquired. These images are obtained to sample, for example, regions in which contrast-enhanced vessels are located (these vessels appear dark in the fluoroscopic images). The images are used to back-project rays as described above in a dense fashion and to look for close intersections—analogous to the approach described above for reconstructing a guidewire. The method then looks for pairs of rays that cross (or nearly cross) taking all combinations of pixels from the two source images (i.e. the dark pixels). The resulting collection of close/exact crossing points is a geometric entity that, when projected, gives rise to the 2×2D images.

Similar to the situation with reconstructing the path of a guidewire, there may be multiple solutions for this reconstruction (path or network determination). Accordingly, a disambiguation process is performed which uses preoperative 3D imaging to help disambiguate the vessel structure—i.e. the pre-operative images act as a constraint for the contemporaneous 3D structure generated by the path crossings.

In other words, the method described above in relation to FIGS. 1-12 reconstructs the 3-dimensional path of a 1-dimensional object, i.e. the guidewire, based on two 2D (X-ray) projections of the object and knowledge of the geometry of the two X-ray C-arms. This approach can be extended to reconstruct topologically more complex objects (networks) in 3D given the respective projections, e.g. determining the blood vessel network within the brain given two contrast-enhanced X-ray projections.

One motivation for the above approach is that it is not possible to acquire specific desired X-ray views of patient anatomy, for example, relating to an aneurysm within the brain. For example, it is not possible to obtain an X-ray view of the brain from the top of the head downwards-a true cranial-caudal view along the long axis of the patient. This is because the required position of the image detector 120 would then conflict with the position of the patient 101 (such as shown in FIG. 1) or potentially with a table on which the patient is positioned. There may also be other positions/orientations which are unavailable because the structure of the detector 120 and/or arm 130 (etc) are unable to accommodate the patient or associated table.

By intra-operatively determining an accurate reconstruction of the contemporaneous 3D structure of target anatomy, then such unavailable views can be generated virtually. In the case of the 1D guidewire reconstruction described above, points are sampled along the length of the respective projections and they are back-projected to the corresponding X-ray sources. In the extended case, points are sampled throughout the dark regions of a network of interest—for example, the set or network of blood vessels.

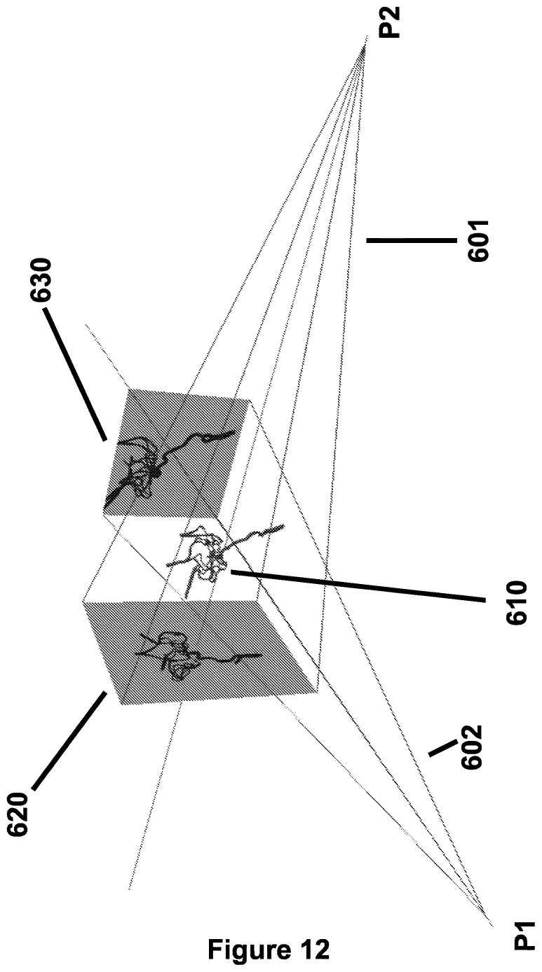

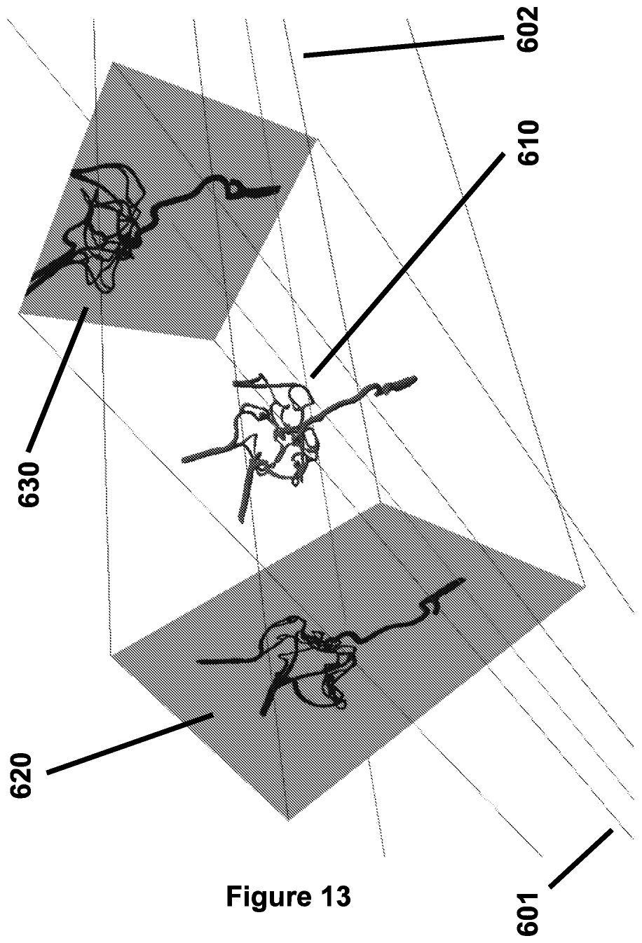

Each of the two respective projections is obtained by extrapolating a line from the X-ray source 114 (which has a known position and orientation) to the selected sample position of the dark region in the image obtained by the X-ray detector 120 (which also has a known position and orientation). This back projection is schematically illustrated in FIGS. 12 and 13, each of which relates to first and second 2-D fluoroscopic images 620, 630 acquired from a 3-D structure or network 610, such as the network of blood vessels in the brain. A set of back-projections is determined for each image, namely back-projections 602 for image 630 to a detector location P1, and back-projections 601 for image 620 to detector location P2. Note that FIGS. 12 and 13 illustrate the same 3D vessel structure 610 but with different X-ray projections. In FIG. 12, the C-arm imaging directions are orthogonal whereas this is not the case in FIG. 13. The change in the position and/or orientation of the fluoroscopic images 620, 630 between FIGS. 12 and 13 may arise for example because the positions P1, P2 of the X-ray source 114 are different. In addition, it will be appreciated that for clarity, FIGS. 12 and 13 show only a few back-projections for each image, however in practice there will typically be thousands of such back-projections determined for each image.

Once all possible pairs of rays from the two (e.g. frontal and lateral) back-projections 601, 602 are generated, locations are identified where a pair of rays cross within a certain distance threshold, and the line of closest approach between the two rays is determined. The midpoint of this line of closest approach, referred to herein as the crossing point, represents the coordinates of the point midway between the places on the back-projected rays that represent the closest approach. These crossing points are used to fill in a lattice spanning the extent of the 3D reconstruction, such that the resulting volumetric (3D) image gives rise to the to the 2D images 620, 630 when projected.

Depending on the shape of anatomy in question and the angles of the C-arms, the resulting volumetric image may represent a non-unique solution, i.e. it may give rise to the correct projections 620, 630, but it may not be a true (complete) representation of the original 3D structure 610. Therefore, as with the 1D guidewire reconstruction discussed above, a disambiguation phase is utilised.

In this case, the topology of the vessel network is known from the acquisition of 3D imaging preoperatively, e.g. during the diagnostic phase. The contemporaneous shape of the vessel network during a procedure may be subject to tissue deformation for example due to gravity or the introduction of instrumentation. By introducing a deformation field, the preoperative image may be mapped onto the reconstructed volume, for example by using a voxel-based comparison metric to determine the parameters of the field via an optimisation process (see for example: [1] D. Rueckert; L. I. Sonoda; C. Hayes; D. L. G. Hill; M. O. Leach; D. J. Hawkes. Nonrigid registration using free-form deformations: application to breast MR images. IEEE Transactions on Medical Imaging, volume 18 (8), pp. 712-721. IEEE, 1999.

Once the 3D shape is recovered such as by use of the deformation field, it can be used to generate an arbitrary number of contemporaneous virtual views. Such virtual views may represent views as described above which cannot be obtained with direct measurement for example because of a potential conflict between the position of the patient and the position of the C-arm fluoroscopy system. These reconstruction techniques, i.e. both the simpler 1D guidewire approach and the approach based on pre-operative imaging, have applications beyond the vessel network within the human brain. For example, such techniques may be applied to other anatomical targets which also exhibit a tree-like structure (network), where 3D preoperative imaging and intraoperative fluoroscopy are typically acquired, e.g. the biliary tree within the liver, and the network of vessels and bronchi within the lungs.

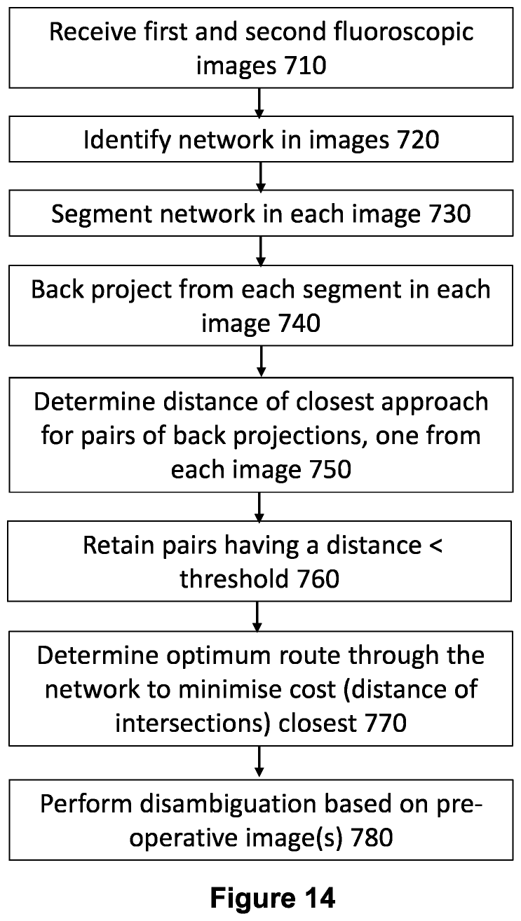

FIG. 14 is a flowchart showing an example of the approach described herein for reconstructing a network. Some portions of this flowchart use the a same or a similar approach as FIG. 11 to reconstruct a guidewire 520.

The procedure starts (710) with the receipt of first and second fluoroscopic images 620, 630. These images are taken by a fluoroscope 100 from two different positions (orientations) at substantially the same time (subject to practical constraints, such as the moving the fluoroscope between the two different positions). Any instrument which is being tracked in the subject is maintained at a constant position within the subject (patient) 101 across both images. The fluoroscope also provides calibrated information with regard to the relative positions between the X-ray source 114 and the image detector 120, and also with regard to the shift in orientation between the first and second images.

At operation 720, the network of interest (whether instrumental or anatomical) is identified in each of the first and second images 620, 630. In other words, a line (or lines) are detected in each image which correspond to the projection or shadow of the network 610 onto the image detector 120 during the acquisition of images 620, 630.

At operation 730, for each image 620, 630, the representation of the network in the images 620, 630 is segmented. In general, a consistent step size or increment is used throughout this segmentation. Note that this constant step size relates to the 2D projection of the network as provided by images 620, 630. The step size is not constant with respect to the corresponding 3D path of the network because the angle between the 3D path and the plane of images 620 and 630 changes as the network curves through or with the anatomy. The step size of the segments (increments) is small enough to ensure an accurate sampling and model of the path of the network.

At operation 740, for each segment in images 620, 630, a back projection is calculated which extends from the segment back to the X-ray source 114. The information for calculating this back projection is available from calibration data such as may be provided with the fluoroscopic images. Accordingly, operation 740 provides a first set of back projections image 620 and a second set of back projections for image 630. The number of back projections corresponds to the number of segments formed across the network path.

At operation 750, intersections are identified between (i) each back projection from image 620 and (ii) each back projection from image 630. Such intersections generally identify 3D spatial locations which are part of the network 610. In practice, an intersection may be indicated at a crossing point when the closest distance between two back projections (one from each image 620, 630) is below a predetermined threshold. These intersections are determined for every pairing of back projections (one from image 620 and one from 630). In broad terms, the 3D path of the network may be determined by an optimisation procedure which uses closest (nearest) distance as a cost (operation 770). There are various techniques available for performing such an optimisation. In practice, a significant proportion of intersections are initially discounted as per operation 760 because their closest distance exceeds the threshold. This simplifies the identification of the 3D network and supports quicker calculation of the 3D path of the network 610, which is particularly beneficial in a real-time, intra-operative environment.

In a final operation 780, a disambiguation may be performed by using a pre-operative image (or images). This disambiguation may be utilised for example when the optimisation does not provide a unique solution. The intra-operative 3D network may be deformed with respect to the pre-operative image (or vice versa) such as by a change in position of the patient, or the insertion of a surgical tool. However, this deformation should not impact the topology (interconnectivity) of the anatomy network. Accordingly, the correct solution for the network is the solution which matches the topology of the pre-operative image(s).

It will be appreciated that the operations shown in FIG. 14 are provided by way of example rather than limitation. In other cases, some of the operations shown in FIG. 14 may be omitted and/or other operations may be added to the processing of FIG. 14 in some implementations. In addition, certain operations may be combined, performed in parallel, or performed in a different order.

The present disclosure further provides a computer system and a computer-implemented method for processing fluoroscopic images to reconstruct network such as a guidewire path. Also provided is a computer program comprising program instructions that when executed on a computer system cause the computing system to perform such a method. The computer program may be provided on a suitable storage medium such as described below. The computer system described herein may be implemented using a combination of hardware and software. The hardware may comprise a standard, general-purpose computing system, or in some implementations, the hardware may include more specialised components, such as graphical processing units (GPUs) and so on to facilitate processing of images by the computer system. The software generally comprises one or more computer programs, e.g. an image processing application, to run on the hardware. These computer programs comprise program instructions which are typically loaded into memory of the computing system for execution by one or more processors to cause the computing system to reconstruct a guidewire as described herein. The computer program may be stored in a non-transitory medium prior to loading into memory, for example, on flash memory, a hard disk drive, etc. The operations of the computer system may be performed sequentially and/or in parallel as appropriate for any given implementation.

Various implementations and examples have been disclosed herein. It will be appreciated that these implementations and examples are not intended to be exhaustive, and the skilled person will be aware of many potential variations and modifications of these implementations and examples that fall within the scope of the present disclosure. It will also be understood that features of particular implementations and examples can typically be incorporated into other implementations and examples (unless the context clearly indicates to the contrary). In summary, the various implementations and examples herein are disclosed by way of illustration rather than limitation, and the scope of the present invention is defined by the appended claims.

1. A computing system configured to:
receive first and second two-dimensional fluoroscopic images acquired at different orientations, each of the images including a representation of a guidewire in a subject;
for each image, divide the representation of the guidewire in that image into a sequence of steps;
for each image, determine for each step in the sequence of steps, a corresponding back projection to an X-ray source used to acquire the fluoroscopic images, thereby generating a first sequential set of back projections for the first image and a second sequential set of back projections for the second image; and
reconstruct a three-dimensional path of the guidewire in the subject based on a sequence of pairs, each pair comprising a first back projection from the first sequential set and a second back projection from the second sequential set, the pairs being selected according to the distance of closest approach between the first back projection and the second back projection.

2. The computing system of claim 1, further configured to identify the representation of the guidewire in each of the first and second images.

3. The computing system of claim 1 or 2, wherein the steps in the sequence of steps for a given image have a constant size.

4. The computing system of any preceding claim, wherein the steps in the first image have the same size as the steps in the second image.

5. The computing system of any preceding claim, wherein the steps in the first and second images are small enough such that each step can be regarded as a straight line segment.

6. The computing system of any preceding claim, wherein for each of the first and second images, the sequence of steps comprises at least 250 steps, preferably at least 1000 steps.

7. The computing system of any preceding claim, further configured to identify a tip of the guidewire in each representation of the guidewire, wherein in each of the first and second images, the tip of the guidewire is selected as a first step in the sequence of steps for that image.

8. The computing system of any preceding claim, wherein pairs having a distance of closest approach between the first back projection and the second back projection which exceeds a threshold are excluded from selection.

9. The computing system of any preceding claim, further configured to generate a node-edge representation in which each pair comprising a first back projection from the first sequential set and a second back projection from the second sequential set forms a node.

10. The computing system of claim 9, wherein each node has a cost based on the distance of closest approach between the first back projection and the second back projection for the pair forming the node.

11. The computing system of claim 9 or 10, further configured to discount nodes which have a cost or distance of closest approach greater than a threshold.

12. The computing system of any of claims 9 to 11, further configured to define edges which link between the nodes to form a route, wherein a first node may be linked by an edge to a second node if the first back projection and/or the second back projection represents a single increment in the respective first and second sequential sets for the second node with respect to the first node.

13. The computing system of any of claims 9 to 12, further configured to determine an optimal route which has a lowest cost based on a summation of the cost for each node in the route, wherein the three-dimensional path of the guidewire in the subject may be reconstructed based on locations of the nodes in the optimal route.

14. The computing system of claim 13, further configured to perform a disambiguation if multiple potential routes are identified.

15. A method of operating a computer system to process fluoroscopic images to reconstruct a guidewire path, the computer-implemented method comprising:
receiving first and second two-dimensional fluoroscopic images acquired at different orientations, each of the images including a representation of a guidewire in a subject;
for each image, dividing the representation of the guidewire in that image into a sequence of steps;
for each image, determining for each step in the sequence of steps, a corresponding back projection to an X-ray source used to acquire the fluoroscopic images, thereby generating a first sequential set of back projections for the first image and a second sequential set of back projections for the second image; and
reconstructing a three-dimensional path of the guidewire in the subject based on a sequence of pairs, each pair comprising a first back projection from the first sequential set and a second back projection from the second sequential set, the pairs being selected according to the distance of closest approach between the first back projection and the second back projection.

16. The method of claim 15, wherein the processing of the fluoroscopic images is performed intra-operatively.

17. A computing system is configured to
receive first and second two-dimensional fluoroscopic images acquired at first and second orientations, wherein each of the images includes a representation of a network in a subject;
for each image, divide the representation of the network in that image into a sequence of steps;
for each image, for each step in the sequence of steps, determine a corresponding back projection to an X-ray source used to acquire the fluoroscopic images, wherein said determining generates a first set of back projections for the first image and a second set of back projections for the second image;
reconstruct a three-dimensional network in the subject based on a sequence of pairs, wherein each pair in the path comprises a first back projection from the first sequential set and a second back projection from the second sequential set and the pairs are selected for having a low distance of closest approach between the first back projection and the second back projection; and
perform a disambiguation by comparing the network to a pre-operative image.

18. The system of claim 17, wherein the disambiguation is performed using a deformation field that links the three-dimensional network to the pre-operative image.

19. A method of operating a computer system to process fluoroscopic images to reconstruct a network, the computer-implemented method comprising:
receiving first and second two-dimensional fluoroscopic images acquired at first and second orientations, wherein each of the images includes a representation of a network in a subject;
for each image, dividing the representation of the network in that image into a sequence of steps;
for each image, for each step in the sequence of steps, determining a corresponding back projection to an X-ray source used to acquire the fluoroscopic images, wherein said determining generates a first set of back projections for the first image and a second set of back projections for the second image;
reconstructing a three-dimensional network in the subject based on a sequence of pairs, wherein each pair in the path comprises a first back projection from the first sequential set and a second back projection from the second sequential set and the pairs are selected for having a low distance of closest approach between the first back projection and the second back projection; and
performing a disambiguation by comparing the network to a pre-operative image.

20. The method of claim 19, wherein the disambiguation is performed using a deformation field that links the three-dimensional network to the pre-operative image.

21. A computer program comprising instructions that when implemented by one or more processors in a computer system causes the computer system to implement the method of claim 15, 16, 19 or 20.

22. A non-transitory storage medium having the computer program of claim 21 stored thereon.
